(19) United States
(12) Patent Application Publication
(10) Pub. No.: US 2018/0140282 A1
(43) Pub. Date: May 24, 2018

(54) ULTRASONIC DIAGNOSTIC APPARATUS AND IMAGE PROCESSING METHOD

(71) Applicant: Hitachi, Ltd., Chiyoda-ku, Tokyo (JP)

(72) Inventors: Takashi TOYOMURA, Tokyo (JP); Masahiro OGINO, Tokyo (JP); Takuma SHIBAHARA, Tokyo (JP); Yoshimi NOGUCHI, Tokyo (JP)

(21) Appl. No.: 15/574,821

(22) PCT Filed: Jun. 3, 2015
(86) PCT No.: PCT/JP2015/066015

§ 371 Date: Nov. 16, 2017

(51) Int. Cl. A61B 8/08; G06T 7/00; G06T 7/60; G06K 9/62

(52) U.S. Cl. CPC A61B 8/5223; A61B 8/5207; A61B 8/0866; G06T 7/0014; G06T 2207/30044; G06K 9/6202; G06K 9/6267; G06T 2207/10132; G06T 2207/30168; G06T 7/60

(57) ABSTRACT

An ultrasonic diagnostic apparatus that extracts a feature to be satisfied as a measurement cross section, classifies the extracted feature according to the degree of importance, and displays and selects an adequate cross-sectional image for each measurement item is provided. The ultrasonic diagnostic apparatus includes an image processing unit 1005 for processing an RF signal from the ultrasonic diagnostic apparatus to generate a cross-sectional image, and a adequacy determination unit 1007 for determining whether the cross-sectional image is adequate, or not, as a measurement image used for measuring a subject included the cross-sectional image acquired by the image processing unit 1005, and has a configuration in which a result determined by the adequacy determination unit 1007 is displayed on a monitor 1006, and presented to an operator.

### TECHNICAL FIELD

The present invention relates to an image processing technique in an ultrasonic diagnostic apparatus.

### BACKGROUND ART

One of fetal diagnoses using the ultrasonic diagnostic apparatus is an examination in which a size of a part of a fetus is measured according to an ultrasonic image and a weight of the fetus is estimated by the following Expression 1.

EFW=1.07BPD³+3.00×10⁻¹AC²×FL [Ex. 1]

where EFW is an estimated fetal weight (g), BPD is a biparietal diameter (cm), AC is abdominal circumference (cm), and FL is a femur length (cm).

In a measurement section image used for a fetal weight estimation, recommended conditions are indicated by Japan Ultrasonic Medical Association in Japan. In a measurement cross section of the biparietal diameter, which is one of objects to be measured, in the Journal of Medical Ultrasonics Vol. 30 No. 3 (2003) “Standardization of ultrasonic fetal measurement and Japanese reference values”, there is a disclosure of “a cross-section in which a midline echo of the fetal head is depicted in a center of the cross-section and a transparent septum pellucidum and a cisterna corpora quadrigemina are depicted”.

Since there is a possibility that target parts may be depicted with different sizes and an estimated fetal weight is miscalculated depending on acquired position and angle of the image of the head measurement section that satisfies the recommended conditions, it is important to accurately acquire a sectional image that satisfies the above features. Patent Literature 1 is available as a prior art for acquiring the measurement section image satisfying the above features without depending on an inspector. Patent Literature 1 discloses that “a luminance spatial distribution feature statistically characterizing a measurement reference image is learned in advance, and a sectional image having the nearest luminance spatial distribution characteristic among multiple sectional images acquired by a cross-section acquisition unit 107 is selected as the measurement reference image”.

### CITATION LIST

### Patent Literature

Patent Literature 1: WO 2012/042808

### SUMMARY OF INVENTION

### Technical Problem

In Patent Literature 1, in the actual measurement, there are restrictions on the position and angle at which the cross-sectional image is acquired by a posture of the fetus in an uterus, and a determination is made based on the whole luminance information on the acquired cross-sectional image. As a result, it is assumed that it is difficult to acquire the cross-sectional image that completely satisfies the features required for the measurement. In other words, there is no high possibility that the acquired image becomes a cross-sectional image most suitable for the measurement by a doctor.

In order to address the above problems, an object of the present invention is to provide an ultrasonic diagnostic apparatus and an image processing method, which are capable of extracting features to be satisfied as a measurement cross-section, classifying the extracted features according to importance, and displaying and selecting a cross-sectional image suitable for each measurement item.

### Solution to Problem

In order to address the above problems, according to the present invention, there is provided an ultrasonic diagnostic apparatus including: an image processing unit that generates an acquired image of a tissue in a subject based on a signal acquired from a probe that transmits and receives ultrasonic waves; an input unit that accepts an instruction from a user; an adequacy determination unit that determines whether the acquired image is adequate as a measurement image used for measuring the subject included in the acquired image, or not; and an output unit that presents to the operator a result determined by the adequacy determination unit.

Further, in order to address the above object, according to the present invention, there is provided an image processing method for an ultrasonic diagnostic apparatus, in which the ultrasonic diagnostic apparatus generates an acquired image of a tissue in a subject based on a signal acquired from a probe that transmits and receives ultrasonic waves, determines whether the acquired image is adequate as a measurement image used for measuring the subject included in the acquired image, or not, and presents a determination result to an operator.

### Advantageous Effects of Invention

According to the present invention, the features to be satisfied as the measurement cross-section can be extracted, the extracted features can be classified according to importance, and the cross-sectional image suitable for each measurement item can be displayed and selected.

### BRIEF DESCRIPTION OF DRAWINGS

FIG. 1 is a block diagram showing an example of a configuration of an ultrasonic diagnostic apparatus according to a first embodiment.

FIG. 2 is a diagram showing an example of a measurement cross-sectional image of a biparietal diameter.

FIG. 3 is a block diagram showing an example of a configuration of an adequacy determination unit according to the first embodiment.

FIG. 4 is a diagram showing an example of a process of creating a template image of a measurement part and components according to the first embodiment.

FIG. 5 is an image diagram for extracting a partial image from an input image according to the first embodiment.

FIG. 6 is an image diagram of a midline detection according to the first embodiment.

FIG. 7 is a positional relationship diagram of components included in a head contour according to the first embodiment.

FIG. 8 is a diagram showing a table for storing a distance between the components included in a measurement target part according to the first embodiment.

FIG. 9 is a diagram showing a table for storing a mean luminance value of pixels forming the components included in the measurement target part according to the first embodiment.

FIG. 10 is a diagram showing a table for storing the weighting factors for use in evaluating whether a condition as the measurement cross-sectional image is satisfied, or not, according to the first embodiment.

FIG. 11 is a diagram showing an example of a screen for presenting a determination result to a user according to the first embodiment.

FIG. 12 is an image diagram for acquiring multiple cross-sectional images by a mechanical scan probe in an ultrasonic diagnostic apparatus according to a second embodiment.

FIG. 13 is a diagram showing a table for storing the degree of adequacy calculated for each cross-sectional image according to a third embodiment.

FIG. 14 is a block diagram showing an example of a configuration of an adequacy determination unit according to the third embodiment.

FIG. 15 is a data flow diagram of the adequacy determination unit according to the third embodiment.

FIG. 16 is an image diagram of partial image extraction according to the third embodiment.

### DESCRIPTION OF EMBODIMENTS

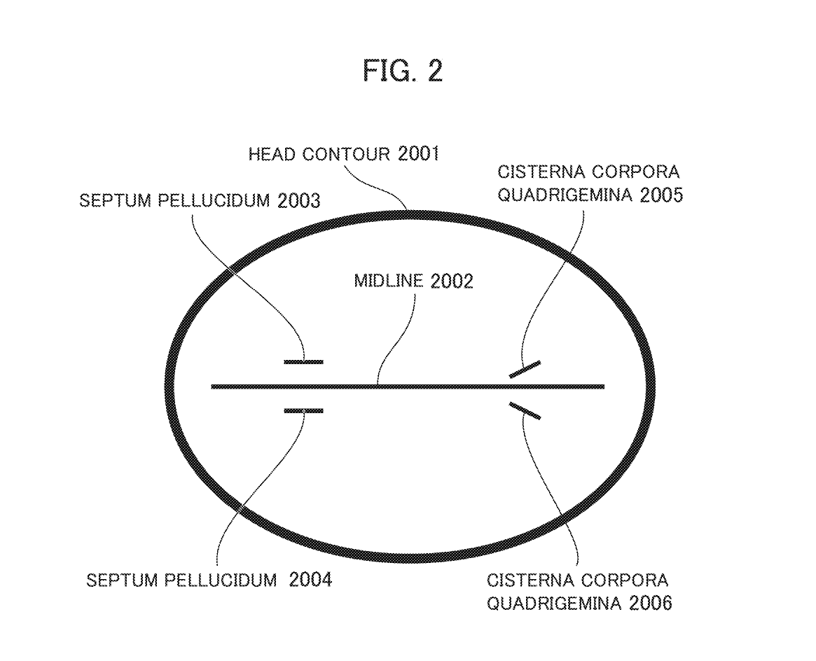

Embodiments of the present invention will be described below with reference to the drawings. In the following embodiments, a head measurement cross-section will be described as an example of a diagnostic target in an ultrasonic diagnostic apparatus, but the present embodiment is applicable to an abdomen measurement cross-section and a femur measurement cross-section in the same way. FIG. 2 shows a head measurement cross-section that satisfies recommended conditions by the Japan Society of Ultrasound Medicine.

As is apparent from the figure, septum pellucidums 2003, 2004, and cisterna corpora quadrigeminas 2005, 2006 are extracted on both sides of the midline 2002 in a head contour 2001.

### First Embodiment

A first embodiment is directed to an ultrasonic diagnostic apparatus configured to include an image processing unit that generates an acquired image of a tissue in a subject based on a signal acquired from a probe that transmits and receives ultrasonic waves, an input unit that accepts an instruction from a user, an adequacy determination unit that determines whether the acquired image is adequate as a measurement image used for measuring the subject included in the acquired image, or not, and an output unit that presents to the operator a result determined by the adequacy determination unit. In addition, the first embodiment is directed to an image processing method for the ultrasonic diagnostic apparatus, which generates an acquired image of a tissue in a subject based on a signal acquired from a probe that transmits and receives ultrasonic waves, determines whether the acquired image is adequate as a measurement image used for measuring the subject included in the acquired image, or not, and presents a determination result to an operator.

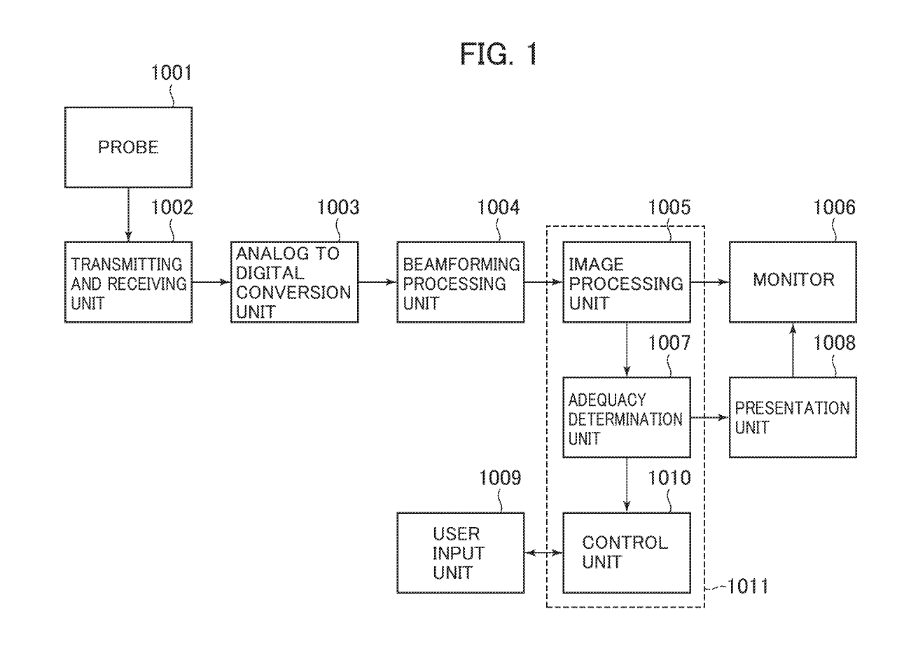

FIG. 1 is a block diagram showing an example of a configuration of an ultrasonic diagnostic apparatus according to a first embodiment. The ultrasonic diagnostic apparatus in FIG. 1 includes a probe 1001 by an ultrasonic transducer for acquiring echo data, a transmitting and receiving unit 1002 that controls a transmission pulse and amplifies a reception echo signal, an analog to digital conversion unit 1003, and a beamforming processing unit 1004 that bundles received echoes from a large number of transducers to add phasing. Also, the ultrasonic diagnostic apparatus includes an image processing unit 1005 that performs dynamic range compression, filter processing, etc., and scan conversion processing on an RF signal from the beamforming processing unit 1004 and generates a cross-sectional image that is an acquired image, a monitor 1006, an adequacy determination unit 1007 for determining whether the image is adequate as an image used for measuring a measurement target part depicted in the cross-sectional image as the acquired image, or not. Further, the ultrasonic diagnostic apparatus includes a user input unit 1009 with a touch panel, a keyboard, a trackball or the like, a control unit 1010 for setting a determination criterion in the determination by the adequacy determination unit 1007, and a presentation unit 1008 that presents to a user a result determined by the adequacy determination unit 1007 with the use of the monitor 1006. In the present specification, the monitor 1006 and the presentation unit 1008 may be collectively referred to as an output unit in some cases.

In the above configuration, when the user operates the probe 1001, the image processing unit 1005 receives the image data through the transmitting and receiving unit 1002, the analog to digital conversion unit 1003, and the beamforming processing unit 1004. The image processing unit 1005 generates a cross-sectional image as the acquired image, and the monitor 1006 displays the cross-sectional image. The image processing unit 1005, the adequacy determination unit 1007, and the control unit 1010 can be realized by a program executed by a central processing unit (CPU) 1011 which is a processing unit of a normal computer. Hereinafter, the adequacy determination unit 1007 and the presentation unit 1008 for presenting the result to the user will be described. The presentation unit 1008 can also be realized by a program of the CPU as with the adequacy determination unit 1007.

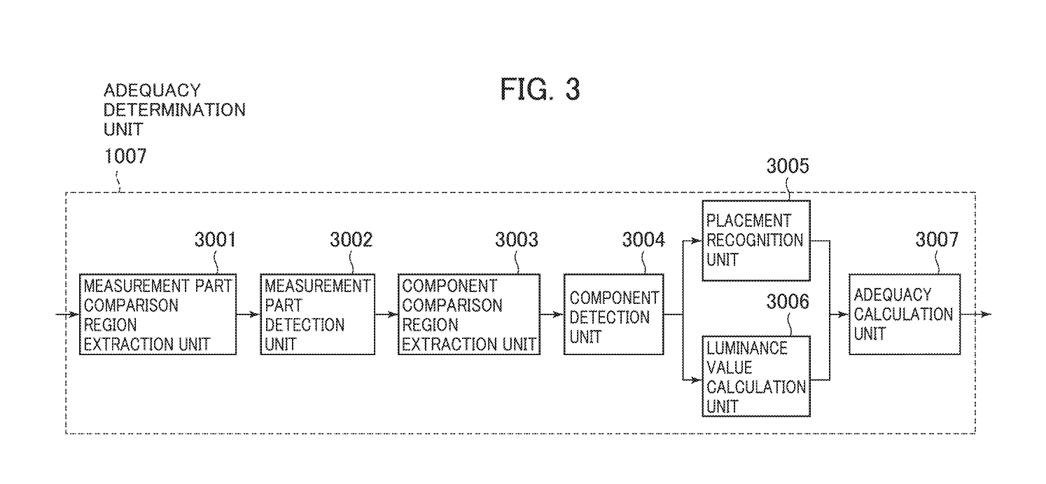

FIG. 3 shows an example of the configuration of the adequacy determination unit 1007 in FIG. 1. As shown in the figure, the adequacy determination unit 1007 includes a measurement part comparison area extraction unit 3001 that extracts first partial images with a predetermined shape and size from the acquired image that is a cross-sectional image received from the image processing unit 1005, a measurement part detection unit 3002 for identifying a first partial image in which the measurement target part is depicted from the multiple first partial images extracted by the measurement part comparison region extraction unit 3001 with the use of edge information, and a component comparison region extraction unit 3003 for extracting a further second partial image with a predetermined shape and size from the first partial image in which the measurement target region is depicted, which is detected by the measurement part detection unit 3002. Also, the adequacy determination unit 1007 includes a component detection unit 3004 for extracting the components included in the measurement target part from the plurality of second partial images extracted by the component comparison area extraction unit 3003 with the use of the edge information, a placement recognition unit 3005 for recognizing a positional relationship of the components, and a luminance value calculation unit 3006 for calculating a mean luminance value for each component. Further, the adequacy determination unit 1007 includes an adequacy calculation unit 3007 for calculating the degree of adequacy indicating whether the cross-sectional image is adequate as an measurement image, or not, with the use of the positional relationship of the components recognized by the placement recognition unit 3005 and the means luminance value for each of the components calculated by the luminance value calculation unit 3006.

As described below, the adequacy determination unit 1007 extracts the first partial images with the predetermined shape and size from the acquired image, identifies the first partial image in which the measurement target part is depicted from the extracted first partial images, extracts second partial images with a predetermined shape and size from the first partial image in which the measurement target part is depicted, extracts the components included in the measurement target part from the extracted multiple second partial images, calculates an evaluation value as a result of checking the positional relationship of the extracted component with a reference value, calculates a mean luminance value for each of the components, and calculates the degree of adequacy indicating whether the acquired image is adequate as the measurement image, or not, with the use of the evaluation value of the component and the mean luminance value of each component.

Specifically, the measurement part detection unit 3002 and the component detection unit 3004 detect the measurement part and the component by template matching. Template images used for the template matching are created in advance from images to be used as the reference of the measurement cross-section and stored in an internal memory of the ultrasonic diagnostic apparatus, a storage unit of the computer, or the like.

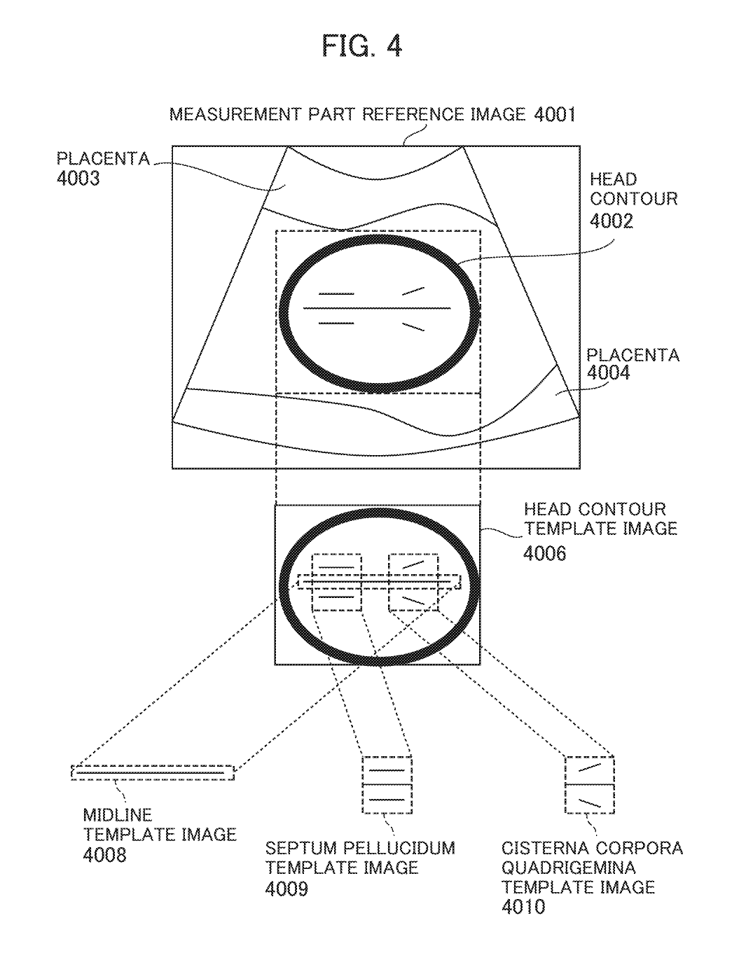

FIG. 4 is a diagram illustrating an example of processing for creating a template image of a measurement part and the components. FIG. 4 shows a measurement cross-section reference image 4001 determined to satisfy the features as the measurement cross section among the images acquired by the ultrasonic diagnostic apparatus. In the measurement cross-section reference image 4001, the head contour 4002 to be measured is depicted together with the tissues inside the uterus such as placentas 4003 and 4004. As described above, in the ultrasonic diagnostic apparatus according to the present embodiment, a head measurement cross-section will be described. In addition, the same processing is performed on an abdomen measurement cross-section and a femur measurement cross-section to enable the determination. The measurement cross-section reference image 4001 may use an image determined by multiple physicians and examination technicians to actually satisfy the features as the measurement cross-section. Alternatively, if an image determined to satisfy the features as the measurement cross-section by the user using the ultrasonic diagnostic apparatus according to the present embodiment may be registered. Further, it is desirable to prepare multiple measurement cross-section reference images 4001 to generate various kinds of template images.

As shown in FIG. 4, only a neighborhood of the head contour is first extracted from the measurement cross-section reference image 4001 to produce a head contour template image 4006. The templates of the components such as the midline are extracted from the head contour template image 4006, and generate a midline template image 4008, a septum pellucidum template image 4009, and a cisterna corpora quadrigemina template image 4010. The septum pellucidum template image 4009 and the cisterna corpora quadrigemina template image 4010 include a part of the midline in an arrangement that traverses in the vicinity of the center. Note that ultrasonic images actually captured vary in size, position, image quality, and the like. Therefore, in order to improve the accuracy of the detection rate by template matching, it is desirable to generate the temperate images of various patterns from the head contour template image 4006, the midline template image 4008, the septum pellucidum template image 4009, and the cisterna corpora quadrigemina template image 4010 which have been generated, by rotating, enlarging, reducing, filtering, edge emphasizing, and the like, respectively, through the program processing of the CPU described above.

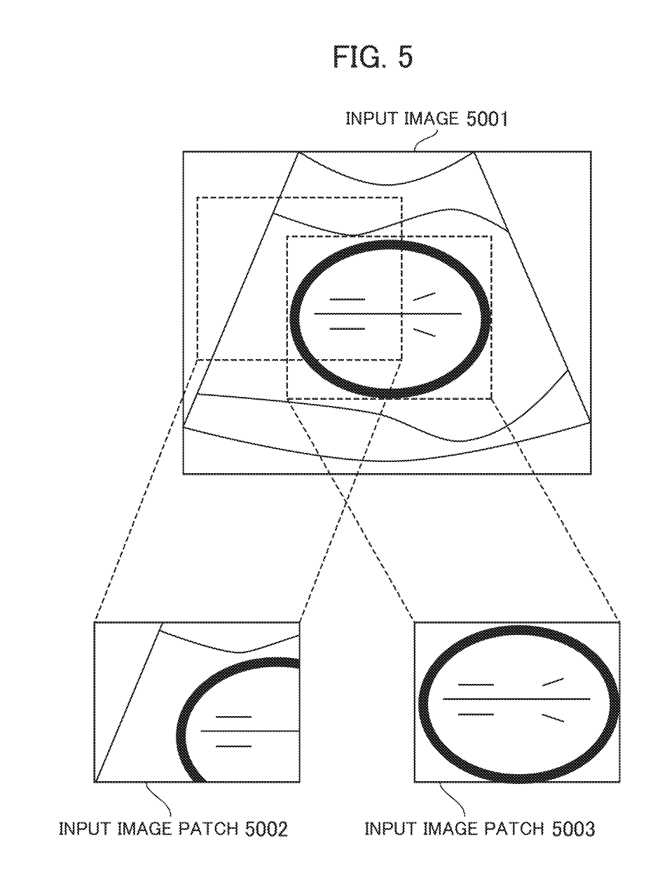

Each processing unit of the adequacy determination unit 1007 shown in FIG. 3 will be described below. The measurement part comparison region extraction unit 3001 extracts multiple first partial images with a predetermined shape and size from one cross-sectional image input from the image processing unit 1005 and outputs the multiple first partial images. FIG. 5 shows a mechanism for extracting input image patches 5002 and 5003 from an input image 5001 with a rectangular shape having a predetermined size. In this example, the input image patches are set to a size large enough to depict the entire measurement part. In FIG. 5, for simplicity of illustration, the first partial images indicated by dotted lines are roughly extracted, but in order to extract the measurement part without any omission, it is desirable to exhaustively extract the first partial images from the entire cross-sectional image.

The measurement part detection unit 3002 detects the input image patch in which the measurement part is depicted by template matching from the input image patches extracted by the measurement part comparison region extraction unit 3001 and outputs the input image patch. In the case of detecting the head contour, the input image patches 5002 and 5003 are sequentially compared with the head contour template image 4006 to calculate the degree of similarity. The degree of similarity is defined as SSD (Sum of Squared Difference) shown in Expression 2 below.

$$SSD = {\sum }_{x = o}^{X - 1} {\sum }_{y = 0}^{Y - 1} {( I ( x , y ) - T ( x , y ) )}^{2} \qquad [ Ex . 2 ]$$

In this example, I(x, y) is a luminance value at coordinates (x, y) of the input image patch, and T(x, y) is the luminance value at the coordinates (x, y) of the template image.

When the input image patch completely matches the head contour template image, the SSD becomes 0. An input image path having the smallest SSD is extracted among all of the input image patches, and output as a head contour extraction patch image. When there is no input image patch whose SSD value is equal to or less than a predetermined value, it is determined that the head contour is not depicted in the input image 5001, and the processing of the present embodiment is terminated. At this time, a fact that the measurement target part could not be detected may be presented to the user by a message or a mark on the monitor 1006, and the user may be urged to input another image.

The degree of similarity between the input image patch and the template image may be defined by SAD (Sum of Absolute Difference), NCC (Normalized Cross-Correlation), ZNCC (Zero-means Normalized Cross-Correlation) instead of SSD. In addition, the measurement part comparison region extraction unit 3001 generates a template image in which a rotation, an enlargement, and a reduction are combined together, thereby being capable of detecting the head contours depicted with various arrangements and sizes. Further, an edge extraction, a noise removal, and so on are applied to both of the template image and the input image patch as preprocessing, thereby being capable of improving a detection accuracy.

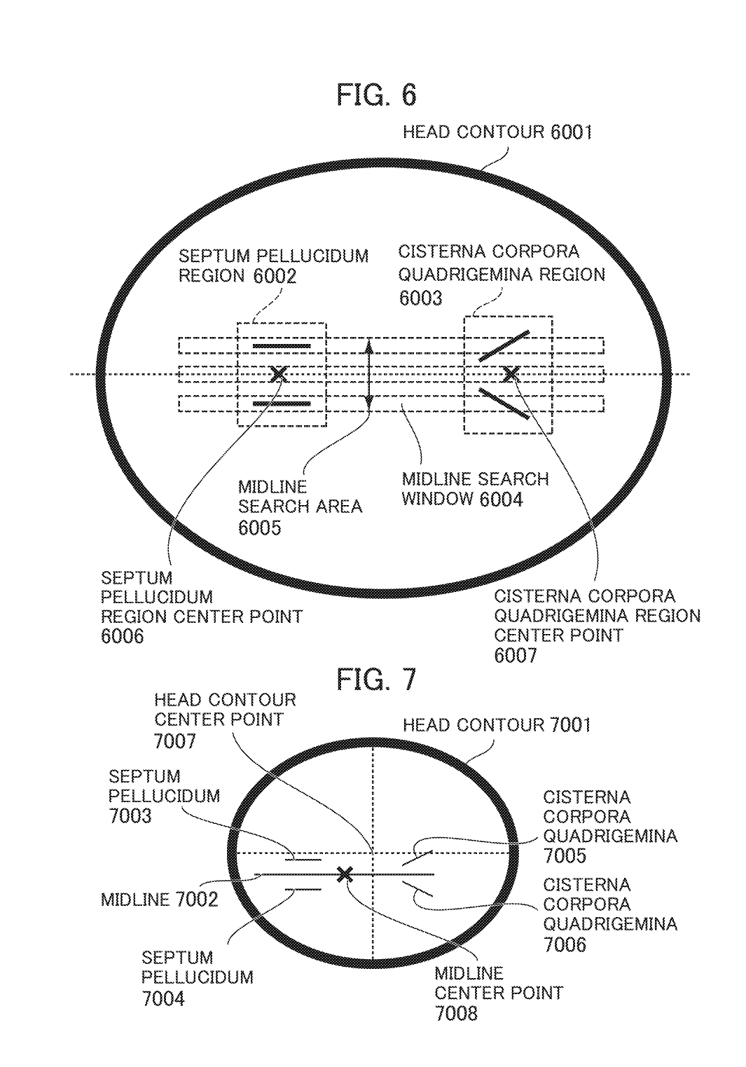

The component comparison region extraction unit 3003 further extracts multiple second partial images with a predetermined shape and size from the input image patch in which the measurement part detected by the measurement part detection unit 3002 is depicted, and outputs the multiple second partial images. In other words, as shown in FIG. 6, the component comparison region extraction unit 3003 extracts the second partial images different according to the shape and size of the component. Hereinafter, the second partial image extracted by the component comparison region extraction unit 3003 is referred to as “a measurement part image patch”. The size of the measurement part image patch is assumed to be 20 pixels×20 pixels as an example so as to sufficiently include the whole of the respective midline, the septum pellucidum, and the cisterna corpora quadrigemina. In addition, the component comparison region extraction unit 3003 may extract multiple measurement part image patches which are the second partial images having different shapes and sizes according to the respective components.

The component detection unit 3004 detects a measurement part image patch in which the component included in the measurement part is depicted by template matching from the measurement part image patches extracted by the component comparison region extraction unit 3003, and outputs the detected measurement part image patch. In the case of detecting the midline, the septum pellucidum, and the cisterna corpora quadrigemina which are located inside the head contours 4002 and 6001, similarly to the processing by the measurement part detection unit 3002, the component detection unit 3004 sequentially compares the measurement part image patch with the midline template image 4008, the septum pellucidum template image 4009, and the cisterna corpora quadrigemina template image 4010 to calculate the respective similarity degrees, and extracts the measurement part image patch having SSD of a predetermined value or lower.

Since the feature amount of the septum pellucidum template image 4009 and the cisterna corpora quadrigemina template image 4010 is larger than that of the midline template image 4008, it is desirable to detect the septum pellucidum template image 4009 and the cisterna corpora quadrigemina template image 4010 prior to the midline. As shown in FIG. 6, if a septum pellucidum region 6002 and a cisterna corpora quadrigemina region 6003 are determined, a straight line passing through a septum pellucidum region center point 6006 that is a center point of the septum pellucidum region and a cisterna corpora quadrigemina region center point 6007 that is a center point of the cisterna corpora quadrigemina region is obtained. A midline search window 6004 is moved in parallel to the straight line, thereby being capable of defining a midline search area 6005, and reducing the amount of calculation. As an example, a size of the midline search window 6004 may be set to twice a distance between the septum pellucidum region center point 6006 and the cisterna corpora quadrigemina region center point 6007.

The placement recognition unit 3005 recognizes a positional relationship of the components identified by the component detection unit 3004. In the case of the head, as shown in FIG. 7, the placement recognition unit 3005 measures a distance between a head contour center point 7007 and a midline center point 7008, and stores the measured distance in a component placement evaluation table to be described below in advance. The head contour center point 7007 is obtained by detecting the head contour by ellipse fitting out of the input image patches in which the head contour detected by the measurement part detection unit 3002 is depicted and calculating an intersection point between a major axis and a minor axis of the ellipse. If the distance is set as a relative value with respect to a length of the minor axis of the ellipse, the evaluation can be performed independently of the size of the head contour depicted in the input image patch.

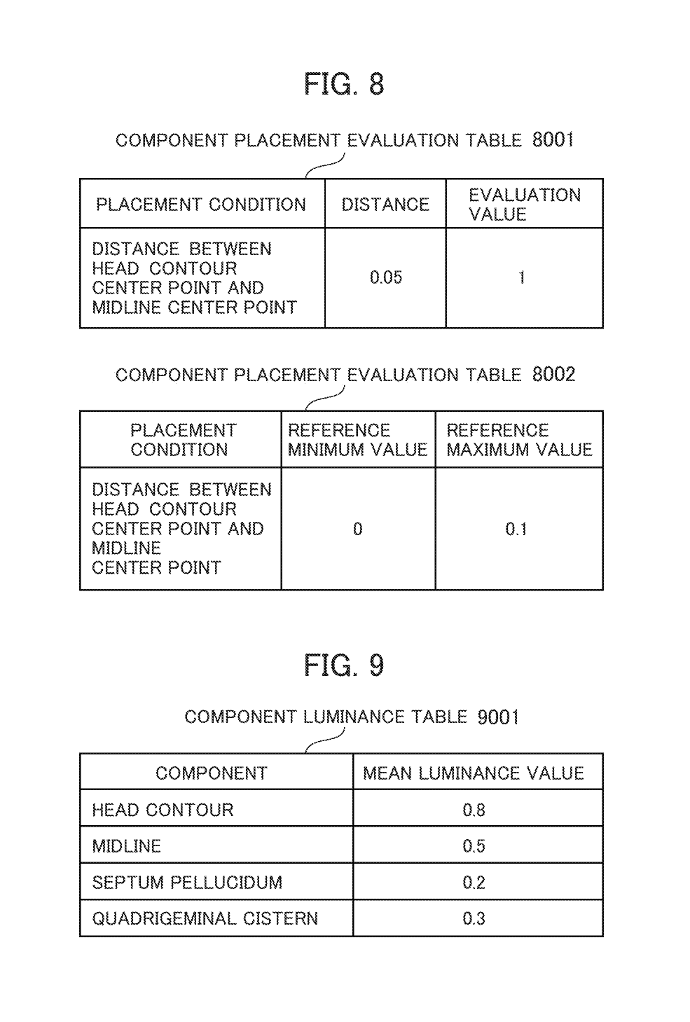

FIG. 8 shows an example of the configuration of a component placement evaluation table and a component placement reference table which are stored in an internal memory of the ultrasonic diagnostic apparatus. A reference value of the distance between the head contour center point 7007 and the midline center point 7008 which are suitable for the measurement cross-section are stored with a minimum value and a maximum value in a component placement reference table 8002 shown in FIG. 8. If the distance stored in the component placement evaluation table 8001 falls within a range from a reference minimum value to a reference maximum value, the evaluation value is set to 1 and if the distance falls outside the range, the evaluation value is set to 0 and stored in the component placement evaluation table 8001.

The luminance value calculation unit 3006 calculates a mean of the luminance values of pixels included in the components specified by the component detection unit 3004, and stores the mean in the component luminance table. FIG. 9 shows an example of the configuration of the component luminance table stored in an internal memory of the ultrasonic diagnostic apparatus, a storage unit of the computer, and the like. In the case of the head, the luminance value calculation unit 3006 calculates a mean luminance value of the pixels on the head contour detected by the placement recognition unit 3005 by ellipse fitting, normalizes the mean luminance value so that a maximum value becomes 1, and stores the normalized mean luminance value in a component luminance table 9001 in advance. The luminance value calculation unit 3006 identifies a midline 7002, septum pellucidums 7003, 7004, and cisterna corpora quadrigeminas 7005, 7006 by straight line detection with the use of Hough transformation for the components and calculates each mean luminance value of the pixels forming each straight line. The means luminance values are normalized as with the head contour and stored in a component luminance table 9001.

The adequacy calculation unit 3007 calculates the degree of adequacy as the measurement cross-section with reference to the component placement evaluation table 8001 and the component luminance table 9001, and outputs the calculated degree of adequacy. The degree of adequacy is represented by the following Expression 3.

$$E = ( \frac{{\sum }_{i = 0}^{M - 1} {a}_{i} {p}_{i}}{M} + \frac{{\sum }_{j = 0}^{N - 1} {b}_{j} {q}_{j}}{N} ) \times \frac{1}{2} \qquad [ Ex . 3 ]$$

In the Expression, E is the degree of adequacy, Pᵢ is each evaluation value stored in the component placement evaluation table 8001, qⱼ is each means luminance value stored in the component luminance table 9001, and aᵢ and bᵢ are weighting factors taking values between 0 and 1. E takes a value between 0 and 1.

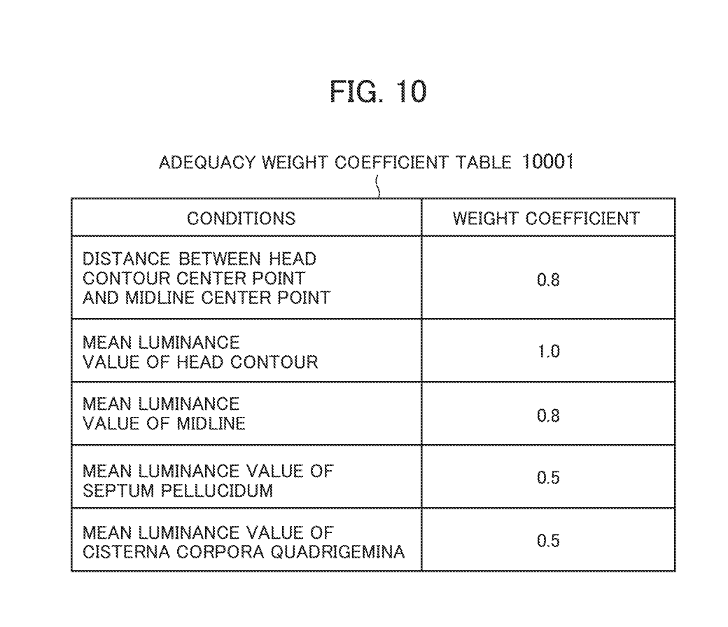

Each weighting factor is stored in advance in an adequacy weighting factor table as shown in FIG. 10. In the case of the head, since it is important that the head contour is clearly depicted for measuring the biparietal diameter, the weighting factor for the mean luminance value of the head contour is set to 1.0. Next, the weighting factor for the distance between the head contour center point and the midline center point, and the mean luminance value of the midline is set to 0.8, and the weighting factor for the means luminance value of the septum pellucidum and the cisterna corpora quadrigemina is set to 0.5. The value of the weighting factor may be designated by the user from the user input unit 1009.

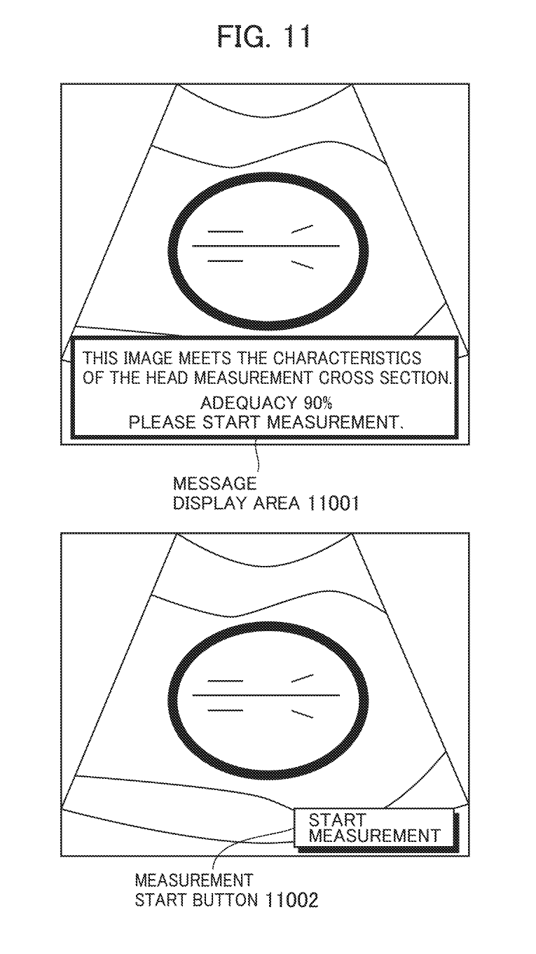

The presentation unit 1008 presents the degree of adequacy calculated by the adequacy calculation unit 3007 to the user through the monitor 1006, and the process is completed. FIG. 11 shows an example of screen display presented to the user. The presenting unit 1008 may express the magnitude of the degree of adequacy with numerical values, marks, and colors as shown in an upper stage of the figure, and may prompt the user to start the measurement. In addition, as shown in a lower stage of the figure, a button to be selected by the user may be validated in order to proceed to the next step such as “start measurement”, for example. When the degree of adequacy is larger than a predetermined value, it is determined that the feature as the measurement section is satisfied, but the user input unit 1009 may designate the predetermined value from the user.

In the ultrasonic diagnostic apparatus according to the present embodiment, the number of weeks of fetus designated by the user through the user input unit 1009 may be used as auxiliary information. Since how to depict a size of the measurement part, the luminance value, and so on differs depending on the number of weeks of fetus, an improvement in detection accuracy can be expected by using the template images of the same fetal week number in the measurement part detection unit 3002 and the component detection unit 3004. Further, the weighting factor of the adequacy weighting factor table 10001 is changed according to the number of weeks of fetus, thereby being capable of more appropriately calculating the degree of adequacy. The number of weeks of fetus may be designated by the user through the user input unit 1009, but the number of weeks of fetus estimated by using the results measured in different parts in advance may be used.

With the ultrasonic diagnostic apparatus according to the first embodiment described in detail above, the features to be satisfied as the measurement cross-section is classified according to importance, and the cross-sectional image that satisfies particularly the features high in importance can be selected.

### Second Embodiment

The present embodiment is directed to an ultrasonic diagnostic apparatus capable of selecting an optimum image as a measurement cross-sectional image when multiple cross-sectional images are input. In other words, the present embodiment is directed to an ultrasonic diagnostic apparatus configured such that an image processing unit generates multiple cross-sectional images, an adequacy determination unit determines whether the multiple cross-sectional images are adequate, or not, and an output unit selects and presents a cross-sectional image determined to be most adequate by the adequacy determination unit. The configuration shown in FIG. 1 described in the first embodiment is used for the apparatus configuration, but a case in which a mechanical scanning type probe is used as a probe 1001 in the present embodiment will be exemplified.

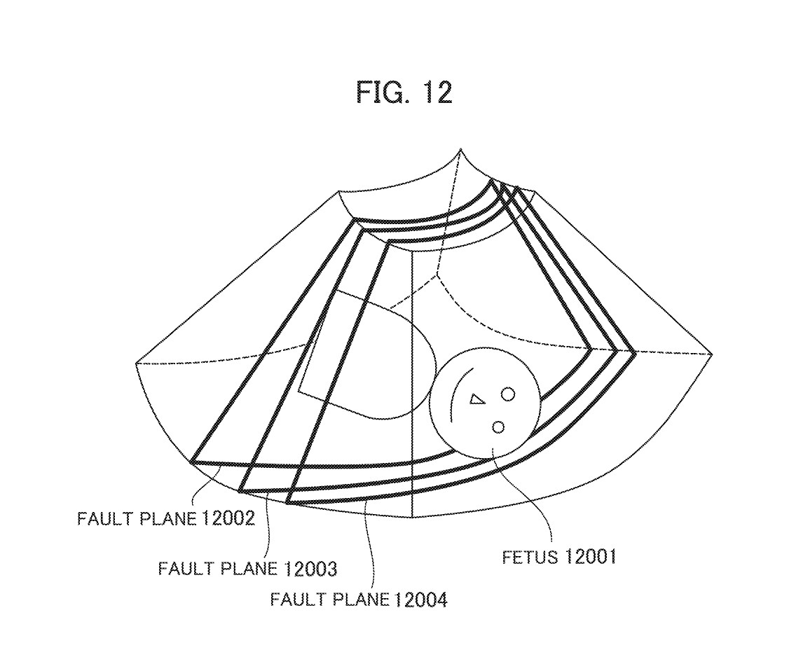

FIG. 12 is an image diagram for acquiring multiple cross-sectional images by a mechanical scan type probe in the ultrasonic diagnostic apparatus. It is needless to say that any method such as a free hand method, a mechanical scan method, a 2D array method, or the like may be used as a method of acquiring multiple cross-sectional image data. An image processing unit 1005 generates a cross-sectional image at each of tomographic planes 12002, 12003, and 12004 with the use of the cross-sectional image data input from a probe 1001 through any one of the methods described above, and stores the generated cross-sectional images in an internal memory of the ultrasonic diagnostic apparatus, a storage unit of a computer, or the like.

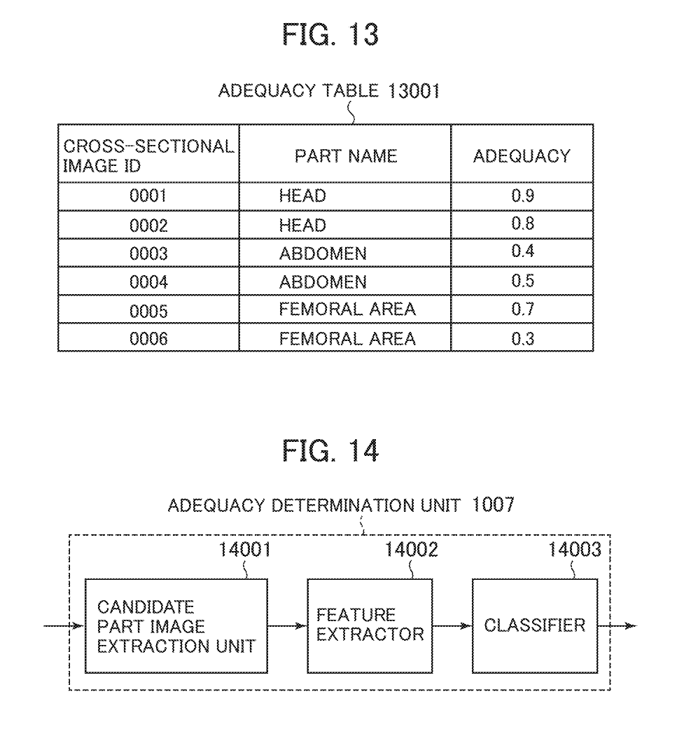

The adequacy determination unit 1007 performs each processing described in the first embodiment on each of the multiple cross-sectional images generated by the image processing unit 1005, and determines the degree of adequacy. The determination result is stored in an adequacy table as shown in FIG. 13. An adequacy table 13001 stores cross-sectional image IDs for identifying cross-sectional images, part names for identifying measurement target parts, and the degree of adequacy of the respective cross-sectional images.

### Third Embodiment

As a third embodiment, a description will be given of an embodiment of a configuration in which feature quantities of a measurement cross-section is identified by machine learning with a smaller processing amount, and determined whether to be adequate, or not. In other words, the present embodiment is directed to an ultrasonic diagnostic apparatus in which the adequacy determination unit includes a candidate partial image extraction unit that extracts a partial image with an arbitrary shape and size from the acquired image, a feature extractor that extracts a feature quantity included in the acquired image from the partial image, and a classifier that identifies and classifies the extracted feature quantity.

In the first embodiment, the components included in the measurement part and the measurement part are extracted by template matching, and the degree of adequacy is determined with the use of the positional relationship of the components and the means luminance value. However, very large throughput is needed for template matching of the multiple cross-sectional images. In the present embodiment, a description will be given of a convolution neural network which extracts and identifies feature quantities from an input image using a machine. The feature quantity may be identified by a Bayesian classification, a k-nearest neighbor algorithm, a support vector machine, or the like with the use of a predetermined index such as a luminance value, an edge, or a gradient. The convolutional neural network has been disclosed in detail, in LECUN et al, “Gradient-Based Learning Applied to Document Recognition,” in Proc. IEEE, vol 86, no 11, November, and so on.

FIG. 14 shows an example of the configuration of the adequacy determining unit 1007 in the case of using machine learning in the apparatus of the present embodiment. Since the other configuration of the apparatus according to the present embodiment has the same configuration as the apparatus configuration of FIG. 1 described in the first embodiment, a description of the other configuration will be omitted. The adequacy determination unit 1007 according to the present embodiment includes a candidate partial image extraction unit 14001 that extracts multiple partial images with an arbitrary shape and size from one cross sectional image generated by the image processing unit 1005, a feature extractor 14002 for extracting a feature quantity included in the image from the extracted partial images, and a classifier 14003 for identifying and classifying the feature quantity.

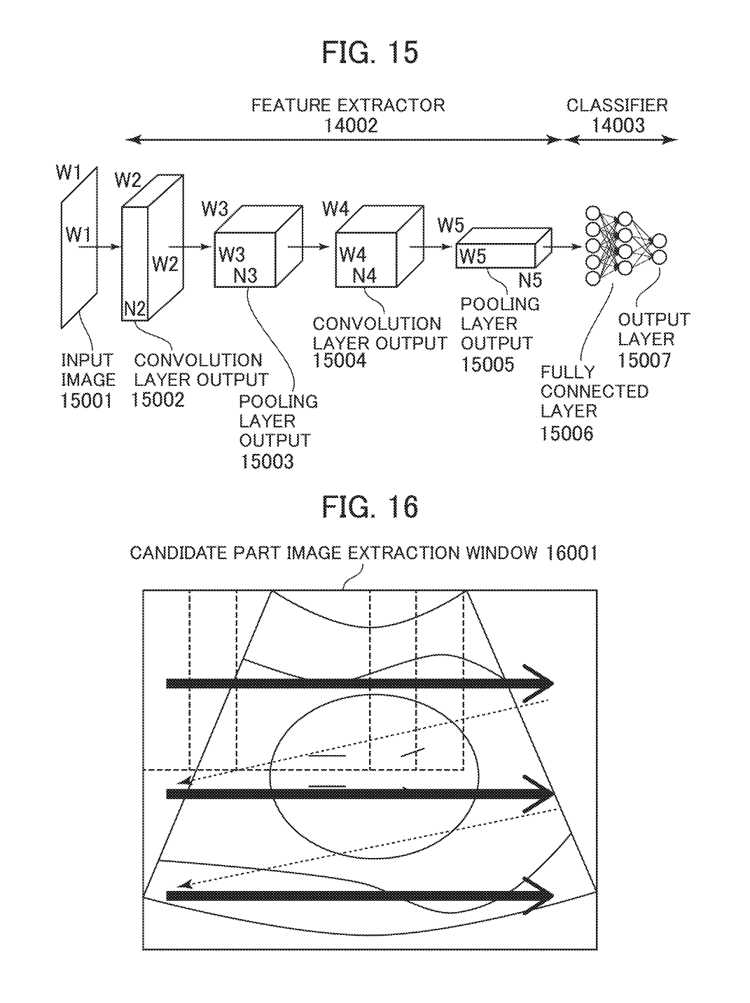

FIG. 15 shows a data flow in the feature extractor 14002 and the classifier 14003 in the case of the convolution neural network. The feature extractor 14002 is configured by connecting multiple convolution layers and multiple pooling layers to each other. The feature extractor 14002 convolutes N2 types of two-dimensional filters of k×k size with respect to an input image 15001 of W1×W1 size, applies an activation function shown by the following Expression 4, and generates, as a convolution layer output 15002, N2 feature maps of W2×W2 size.

$$f ( x ) = \frac{1}{1 + {e}^{- x}} \qquad [ Ex . 4 ]$$

In this expression, f is an activation function, and x is an output value of a two-dimensional filter.

Although Expression 4 is a sigmoid function, a rectified linear unit or Maxout may be used as the activation function. The purpose of the convolution layer is to obtain local features by blurring a part of the input image or emphasizing the edge. In the case of head measurement, as an example, W1 is set to 200 pixels, k is set to 5 pixels, and W2 is set to 196 pixels. In the next pooling layer, a maximum pooling shown in Expression 5 is applied to the feature map generated by the convolution layer to generate a pooling layer output 15003 of W3×W3 size.

$${y}^{′} = {max}_{i ∈ P} {y}_{i} \qquad [ Ex . 5 ]$$

in this example, P is a region of an s×s size extracted from the feature map at an arbitrary position, yᵢ is a luminance value of each pixel included in the extracted region, and y′ is a luminance value of the pooling layer output.

In the case of the head measurement, s is set to 2 pixels as an example. As a pooling method, mean pooling or the like may be used. The feature map is reduced by the pooling layer, and robustness can be ensured against minute position changes of features in the image. The similar processing is performed also on the convolution layer and the pooling layer at a post stage to generate a pooling layer output 15005. The classifier 14003 is a neural network configured by a fully connected layer 15006 and an output layer 15007, and outputs a classification result as to whether the input image satisfies the feature as the measurement cross-section, or not. Units of each layer are completely connected to each other, and for example, one unit of the output layer and a unit of an intermediate layer at a preceding stage have a relationship expressed by the following Expression 6.

$${O}_{i} = g ( {\sum }_{j = 0}^{N - 1} {c}_{ij} {r}_{j} - d ) \qquad [ Ex . 6 ]$$

In this case, Oᵢ is an output value of an ith unit of the output layer, g is an activation function, N is the number of units of the intermediate layer, Cᵢⱼ is a weighting factor between the jth unit of the intermediate layer and the ith unit of the output layer, rⱼ is an output value of the jth unit of the intermediate layer, and d is a bias. cᵢⱼ and d are updated by a learning process to be described later so as to be able to discriminate whether the feature as the measurement cross-section is satisfied, or not.

Next, a process of causing the convolution neural network of FIG. 15 to perform learning will be described. In the convolution neural network according to the present embodiment, supervised learning is performed. As the learning data, multiple input images normalized to a W1×W1 size and labels as to whether each input image satisfies the feature as the measurement cross-section, or not, are prepared. As the input image, not only the measurement cross-section reference image but also a sufficient number of images that do not satisfy the feature as the measurement cross-section such as an image of an intrauterine tissue such as a placenta and the head contour image where the midline is not depicted need to be prepared. Learning is performed by updating the weights and biases of a two-dimensional filter of the convolution layer and the fully connected layer with the use of an error back propagation method so that an error between the discrimination result obtained for the input image and the label prepared as learning data becomes small. Learning is completed by performing the above processing on all of input images prepared as learning data.

Next, a process of determining whether the cross-sectional image satisfies the feature as the measurement cross-section, or not, with the use of the convolution neural network which has completed the learning will be described. The candidate partial image extracting unit 14001 exhaustively extracts the partial images from the entire input cross-sectional image and outputs the partial images. As indicated by arrow lines in FIG. 16, a candidate partial image extraction window 16001 is finely moved from an upper left to a lower right of the cross-sectional image to extract partial images. The feature extractor 14002 and the classifier 14003 successively extract and classify the candidate partial images generated by the candidate partial image extraction unit 14001, and the classifier 14003 outputs a likelihood appropriate for the measurement cross-section and an inappropriate likelihood. For the cross-sectional image determined by the classifier 14003 to satisfy the feature as the measurement cross-section, an output value of the classifier 14003 is stored in the adequacy table 13001 as the degree of adequacy. The presentation unit 1008 refers to the adequacy level table 13001 and presents to the user a cross-sectional image having the maximum degree of adequacy among the cross-sectional images including the measurement target part. The presentation unit 1008 may indicate a cross-sectional image having the maximum i-degree of adequacy with the use of a message in the same manner as shown in the upper stage of FIG. 11, or may display a list of the multiple cross-sectional images and may indicate a cross-sectional image having the maximum degree of adequacy among those multiple cross-sectional images with a message, a mark, or framing.

As described above, according to the apparatus of the present embodiment, since an optimum cross-sectional image is selected as a measurement cross-sectional image from the multiple cross-sectional images, a trouble of the user repeatedly acquiring the image and confirming the calculation result of the degree of adequacy can be omitted.

The respective embodiments of the present invention have been described above. However, the present invention includes various modified examples. For example, in the above-mentioned embodiments, in order to easily understand the present invention, the specific configurations are described. However, the present invention does not always provide all of the configurations described above. For example, in the embodiments described above, the ultrasonic diagnostic apparatus including the probe or the like has been described as an example. However, the present invention can be applied to a signal processing device that executes processing subsequent to the image processing unit on the storage data of the storage device in which the obtained RF signal and so on are accumulated. Also, a part of one configuration example can be replaced with another configuration example, and the configuration of one embodiment can be added with the configuration of another embodiment. Also, in a part of the respective configuration examples, another configuration can be added, deleted, or replaced.

Furthermore, an example in which a program that realizes parts or all of the above-described respective configurations, functions, processors, controllers, and so on is created has been described. Alternatively, parts and all of those configurations may be realized by hardware, for example, designed by an integrated circuit.

### LIST OF REFERENCE SIGNS

1001 probe
1002 transmitting and receiving unit
1003 analog to digital conversion unit
1004 beamforming processing unit
1005 image processing unit
1006 monitor
1007 adequacy determination unit
1008 presentation unit
1009 user input unit
1010 control unit
1011 CPU
3001 measured part comparison region extraction unit
3002 measured part detection unit
3003 component comparison region extraction unit
3004 component detection unit
3005 placement recognition unit
3006 luminance value calculation unit
3007 adequacy calculation unit
14001 candidate partial image extraction unit
14002 feature extraction unit
14003 classifier

1. An ultrasonic diagnostic apparatus comprising:
an image processing unit that generates an acquired image of a tissue in a subject based on a signal acquired from a probe that transmits and receives ultrasonic waves;
an input unit that accepts an instruction from a user;
an adequacy determination unit that determines whether the acquired image is adequate as a measurement image used for measuring the subject included in the acquired image, or not; and
an output unit that presents to the operator a result determined by the adequacy determination unit.

2. The ultrasonic diagnostic apparatus according to claim 1, wherein the adequacy determination unit comprises:
a measurement part comparison region extraction unit that extracts first partial images with a predetermined shape and size from the acquired image;
a measurement part detection unit that identifies an image in which a measurement target part is depicted from the first partial images extracted by the measurement part comparison region extraction unit;
a component comparison region extraction unit that extracts second partial images with a predetermined shape and size from the first partial image in which the measurement target part is depicted;
a component detection unit that extracts a component included in the measurement target part from a plurality of the second partial images extracted by the component comparison area extraction unit;
a placement recognition unit that calculates an evaluation value as a result of collating a positional relationship of the extracted components with a reference value;
a luminance value calculation unit that calculates a mean luminance value for each of the components; and
an adequacy calculation unit that calculates a degree of adequacy indicating whether the acquired image is adequate as a measurement image, or not, with the use of the evaluation value of the component and the mean luminance value of each of the components.

3. The ultrasonic diagnostic apparatus according to claim 2, wherein the adequacy calculation unit multiplies the evaluation value of the component and the mean luminance value for each of the components by respective weighting factors to calculate the degree of adequacy.

4. The ultrasonic diagnostic apparatus according to claim 3, wherein the weighting factors can be varied based on an instruction from the input unit.

5. The ultrasonic diagnostic apparatus according to claim 1, the adequacy determination unit comprises:
a candidate partial image extraction unit that extracts a partial image with an arbitrary shape and size from the acquired image;
a feature extractor that extracts a feature quantity included in the acquired image from the partial image; and
a classifier that identifies and classifies the extracted feature quantity.

6. The ultrasonic diagnostic apparatus according to claim 1, wherein the image processing unit generates a plurality of cross-sectional images,
the adequacy determination unit determines whether the plurality of cross-sectional images are adequate, or not, and
the output unit selects and presents a cross-sectional image determined to be most adequate by the adequacy determination unit.

7. An image processing method for an ultrasonic diagnostic apparatus, wherein
the ultrasonic diagnostic apparatus
generates an acquired image of a tissue in a subject based on a signal acquired from a probe that transmits and receives ultrasonic waves,
determines whether the acquired image is adequate as a measurement image used for measuring the subject included in the acquired image, or not, and
presents a determination result to an operator.

8. The image processing method according to claim 7, wherein the ultrasonic diagnostic apparatus
extracts first partial images with a predetermined shape and size from the acquired image,
identifies an image in which a measurement target part is depicted from the extracted first partial images,
extracts second partial images with a predetermined shape and size from the first partial image in which the measurement target part is depicted,
extracts a component included in the measurement target part from a plurality of the extracted second partial images,
calculates an evaluation value as a result of collating a positional relationship of the extracted components with a reference value,
calculates a mean luminance value for each of the components, and
calculates the degree of adequacy indicating whether the acquired image is adequate as a measurement image, or not, with the use of the evaluation value of the component and the mean luminance value of each of the components.

9. The image processing method according to claim 8, wherein the ultrasonic diagnostic apparatus multiplies the evaluation value of the component and the mean luminance value for each of the components by respective weighting factors to calculate the degree of adequacy.

10. The image processing method according to claim 9, wherein the ultrasonic diagnostic apparatus can vary the weighting factors based on a user's instruction from an input unit.

11. The image processing method according to claim 7, wherein the ultrasonic diagnostic apparatus
extracts a partial image with an arbitrary shape and size from the acquired image,
extracts a feature quantity included in the acquired image from the extracted partial image, and
identifies and classifies the extracted feature quantity to determine whether the acquired image is adequate, or not.

12. The image processing method according to claim 7, wherein the ultrasonic diagnostic apparatus
generates a plurality of cross-sectional images,
determines whether the plurality of cross-sectional images are adequate, or not, and
selects a cross-sectional image determined to be most adequate and presents the selected cross-sectional image to an output unit.
